(19) United States
(12) Patent Application Publication
(10) Pub. No.: US 2016/0143544 A1
(43) Pub. Date: May 26, 2016

(54) System for Calculating Biological Information Under Exercise Load, Biological Information Calculation Method, and Portable Information Terminal

(71) Applicant: HITACHI, LTD., Chiyoda-ku, Tokyo (JP)

(72) Inventor: Takeshi TANAKA, Tokyo (JP)

(21) Appl. No.: 14/392,156

(22) PCT Filed: Jun. 27, 2013
(86) PCT No.: PCT/JP2013/067719

§ 371 Date: Dec. 23, 2015

(51) Int. Cl. A61B 5/0205; A61B 5/00

(52) U.S. Cl. CPC A61B 5/0205; A61B 5/0015

(57) ABSTRACT

A system for calculating biological information under exercise load, including a sensor device for measuring a motion of a user, a portable heart rate sensor for measuring a heart rate of a user, and a server including a biological information calculation function, wherein the system measures a motion of a user under exercise load on the user, measures the heart rate of the user after the stop of the exercise load, measures a temporal difference between the stop of the exercise load and measurement of the heart rate, estimates a drop in the heart rate based on the temporal difference and finds an estimated heart rate immediately before the stop of exercise of the user, and calculates biological information of the user under the exercise load based on the motion of the user under the exercise load and the estimated heart rate.

### TECHNICAL FIELD

The present invention relates to a system for calculating biological information under person's exercise load, a biological information calculation method, and a portable information terminal, and particularly to a technique for calculating biological information by use of a wearable simplified sensor device and a portable information terminal capable of measuring or inputting a pulse rate.

### BACKGROUND ART

It is known that fitness by daily exercise leads to prevention of lifestyle-related diseases such as diabetes and hypertension. In order to quantitatively grasp an increased physical strength effect due to exercise, it is most reasonable to measure maximum oxygen uptake (VO₂max) indicating whole-body stamina among physical strengths. On the other hand, in order to accurately measure maximum oxygen uptake, high-load exercises and large devices such as breathalyzer device are required, but are not suitable for daily simplified measurement.

PTL 1 discloses therein a measurement system for detecting a steady exercise state for more than a certain time suitable for estimating maximum oxygen uptake (VO₂max) by an acceleration sensor or the like, and estimating maximum oxygen uptake based on an exercise intensity at that time and a heart rate during exercise measured by a pulse wave sensor.

PTL 2 discloses therein a measurement system for estimating the exercise load amount based on a vertical acceleration during walking measured by an acceleration, and estimating physical strength by regression analysis based on the exercise load amount and a heart rate during exercise measured by a pulse rate sensor.

### CITATION LIST

### Patent Literatures

PTL 1: JP 2011-200557 A

PTL 2: JP 2002-253538 A

### SUMMARY OF INVENTION

### Technical Problem

In order to accurately measure a heart rate under exercise load by a heart rate sensor for estimating physical strength at a high accuracy, a chest belt or electrode of a heart rate sensor needs to be directly fixed on the skin of a user. For example, however, general users, who do not play sports, always do not want to wear a heart rate sensor needing a complicated operation, and thus a heart rate cannot be measured in a simple manner under low load such as during walking or on the way to work.

On the other hand, PTL 1 discloses a measurement system assuming that a pulse wave sensor or acceleration sensor is mounted on the wrist of a user thereby to measure motions of the body and a heart rate during measurement walking or running. PTL 2 discloses therein a measurement system assuming that a pulse rate sensor is mounted on either ear of a user and an acceleration sensor is separately mounted on the user thereby to use a pulse rate and an acceleration measured by the sensors.

With a simplified pulse rate sensor wearable on a wrist or an ear of a user for measurement, however, a heart rate (or pulse rate) is difficult to measure at a high accuracy due to noises caused by external light or electrode offset by body motions during exercise. When a simplified pulse rate sensor, is used, measurements need to be made at rest after exercise is stopped. On one hand, with the heart rate measured after exercise is stopped, a heart rate drops over time after exercise load, a heart rate corresponding to exercise strength under exercise load cannot be obtained, and an accuracy of estimating person's physical strength such as maximum oxygen uptake (VO₂max) lowers. On the other hand, a device to be mounted on the wrist or the like of a user for acquiring a heart rate (or pulse rate) at a high accuracy is also possible but inevitably leads to a special precision device for higher accuracy, and general user cannot easily use it.

It is an object of the present invention to provide a technique capable of estimating person's physical strength under low load by use of a simplified pulse rate sensor or cardiac sensor in order to estimate physical strength based on person's exercise data and heart rate under exercise load.

### Solution to Problem

One representative example of the present invention is as follows.

A system for calculating biological information under exercise load according to the present invention includes a sensor device for measuring a motion of a user, a portable heart rate sensor for measuring a heart rate of a user, and a server including a biological information calculation function, wherein the system measures a motion of a user under exercise load on the user, measures the heart rate of the user after the stop of the exercise load, measures a temporal difference between the stop of the exercise load and measurement of the heart rate, estimates a drop in the heart rate based on the temporal difference and finds an estimated heart rate immediately before the stop of exercise of the user, and calculates biological information of the user under the exercise load based on the motion of the user under the exercise load and the estimated heart rate.

### Advantageous Effects of Invention

According to the present invention, it is possible to accurately measure a heart rate during exercise and to estimate biological information under exercise load such as maximum oxygen uptake (VO₂max) at a high accuracy based on the heart rate data and body motion data measured by an acceleration sensor even by use of an inexpensive and simplified pulse rate sensor or cardiac sensor not suitable for measurement during exercise.

### BRIEF DESCRIPTION OF DRAWINGS

FIG. 1 is a diagram illustrating an entire structure of a system for calculating biological information under exercise load according to a first exemplary embodiment of the present invention.

FIG. 2 is a structure diagram of a sensor device according to the first exemplary embodiment.

FIG. 3 is a structure diagram of a portable information terminal according to the first exemplary embodiment.

FIG. 4 is a flowchart illustrating an outline of a processing of calculating biological information under exercise load according to the first exemplary embodiment.

FIG. 5 illustrates an exemplary structure of acceleration data stored in a server according to the first exemplary embodiment.

FIG. 6 illustrates an exemplary structure of heart rate data stored in the server according to the first exemplary embodiment.

FIG. 7 illustrates an exemplary structure of exercise characteristic amount data stored in the server according to the first exemplary embodiment.

FIG. 8 illustrates an exemplary structure of body characteristic amount data stored in the server according to the first exemplary embodiment.

FIG. 9 is a graph of heart rate drop indicating a principle of correcting heart rate.

FIG. 10 is a graph of heart rate drop indicating a principle of correcting heart rate.

FIG. 11 illustrates an exemplary structure of heart rate drop data stored in the server according to the first exemplary embodiment.

FIG. 12 illustrates an exemplary structure of maximum oxygen uptake data stored in the server according to the first exemplary embodiment.

FIG. 13A is a flowchart illustrating the processing of calculating biological information under exercise load in detail according to the first exemplary embodiment.

FIG. 13B is a flowchart illustrating the processing of calculating biological information under exercise load in detail according to the first exemplary embodiment.

FIG. 14 is a graph illustrating a typical principle of measuring maximum oxygen uptake (VO₂max).

FIG. 15 is a time sequence graph illustrating a principle of correcting heart rate according to the first exemplary embodiment.

FIG. 16 is a graph illustrating a principle of estimating maximum oxygen uptake (VO₂max) according to the first exemplary embodiment.

FIG. 17 illustrates exemplary screen display of a portable information terminal according to the first exemplary embodiment.

FIG. 18 illustrates exemplary screen display of the portable information terminal according to the first exemplary embodiment.

FIG. 19 illustrates exemplary screen display of the portable information terminal according to the first exemplary embodiment.

### DESCRIPTION OF EMBODIMENTS

According to the present invention, a heart rate during exercise is estimated and corrected based on a heart rate after exercise in a technique for estimating physical strength based on person's exercise data and post-exercise heart rate or pulse rate by use of an ultracompact sensor device wearable on a person body for measuring motions and a portable information terminal capable of measuring or inputting a heart rate. That is, according to the present invention, body motions during exercise are measured, the exercise is stopped, and then a heart rate is measured at rest without any noise influence due to body motions by use of a non-complicated portable information terminal or a simplified cardiac sensor or pulse rate sensor unlike sensors including chest band directly fixed on the skin. Then, a temporal difference between the stop of the exercise and the measurement of a heart rate is measured, and a drop from a heart rate immediately before the end of the exercise is estimated based on the temporal difference, and is converted and corrected to a heart rate immediately before the end of the exercise. Thereby, person's physical strength under low load is estimated at a higher accuracy.

A system for calculating biological information under exercise load according to one exemplary embodiment of the present invention will be described below with reference to the drawings.

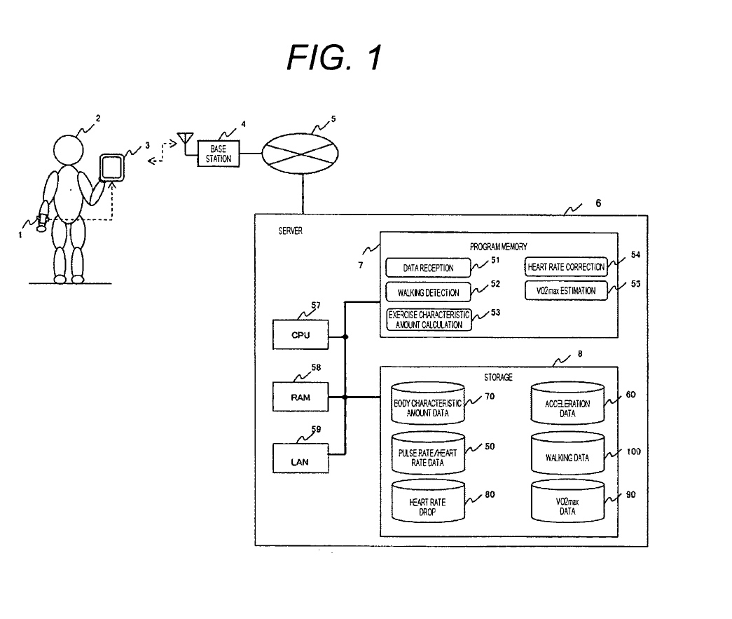

FIG. 1 illustrates an entire structure of main components in a system for calculating biological information under exercise load according to a first exemplary embodiment of the present invention. The system for calculating biological information under exercise load is configured of a sensor device 1 wearable on the body of a user 2, a portable information terminal 3 carried by the user 2 for communicating with the sensor device 1, and a server 6 for communicating with the portable information terminal 3 via a wireless base station 4 for cell phone communication or the like connected to the Internet 5. The system for calculating biological information under exercise load collects sensor data from the ultracompact and low-power consumption sensor device 1 capable of being always carried on the user 2, though with low-speed processing, and the portable information terminal 3 to the large-sized server 6 with high processing capability and large storage capacity, thereby making large-scaled analysis at a high speed.

The sensor device 1, which is carried on the user 2 always or during main activity time of the day, measures motions or biological information of the user 2. The sensor device 1 transmits the measured information to the portable information terminal 3 via short-distance wireless communication such as Bluetooth (trademark) or a wired communication means such as USB. The portable information terminal 3 records the received measurement data. Further, the portable information terminal 3 measures and records therein biological information of the user 2 by its built-in sensor or camera. That is, the portable information terminal 3 includes a function as a simplified pulse rate sensor easily usable by the user with the built-in camera and application programs according to the present invention described below. In this case, the function added as a pulse rate sensor by the application programs can be provided at low cost.

The portable information terminal 3 is connectable to the base station 4 at home or outside home by a wide-area wireless communication means used for cell phones and the like, or wireless LAN (local area network). Thereby, the server 6 connected to the Internet 5, and the portable information terminal 3 can mutually transmit information measured by the sensor device 1, information measured by the portable information terminal 3, or information analyzed by or accumulated in the server 6.

The server 6 is configured of a CPU 57, a RAM 58, a LAN (local area network) communication unit 59 connected to the Internet 5 for communication, a program memory 20 for recording therein a plurality of computer programs for controlling the server 6 and making necessary calculations (such as a data reception program 51, a walking detection program 52, an exercise characteristic amount calculation program 53, a heart rate correction program 54, and a maximum oxygen uptake (VO₂max) estimation program 55), and a storage 36 as a large-capacity storage device for recording data measured by the sensor device 1 or the portable information terminal 3, or results of analyzed data, and can perform a large-scaled analysis processing by use of large-capacity data by the CPU 57 capable of calculations at a higher speed than the sensor device 1 or the portable information terminal 3. The computer programs are executed by the CPU 57 in the server 6 to cause the server (computer) to function as a data reception means, a walking detection means, an exercise characteristic amount calculation means, a heart rate correction means, and a maximum oxygen uptake (VO₂max) estimation means.

The server 6 connected to the Internet 5 receives the data transmitted from the portable information terminal 3 by the data reception program 51 and records it in acceleration data 60, heart rate data 50, and body characteristic amount data 70 in the storage 36. The walking detection program 52 analyzes the acceleration data 60, detects a time interval in which consumed oxygen is balanced with oxygen uptake and walking lasts for more than three minutes at a constant pace by a well-known walking detection algorithm, and records a user ID as a predefined user identifier as well as the start time and the end time of walking in walking data 100. The exercise characteristic amount in the walking interval detected by the walking detection program 52 is calculated from the acceleration data 60 to be recorded in the same interval in the walking data 100 by the exercise characteristic amount calculation program 53. The exercise characteristic amount is average or variance of three axes (x, y, z) of acceleration, walking pitch (duration per step) determined based on acceleration, and the like. An average of acceleration indicates an orientation in which acceleration is mainly influenced by gravity acceleration, or a posture, and a variance indicates a strength of vibration of acceleration.

The heart rate correction program 54 is directed for converting and correcting a heart rate of the user measured at a short time after a predetermined time of applying exercise loads on the user, for example, exercise such as aerobic exercise into a heart rate during exercise immediately before the end of the exercise of the user. Needless to say, exercise loads on the user may be other types of loads such as jogging and cycling. In the present exemplary embodiment, “walking” with light load capable of easily performed by the user is employed as exercise load.

Any means capable of being used as a simplified pulse rate sensor or cardiac sensor and including a user interface function and a communication function may be employed as a means for measuring a heart rate, not limited to the portable information terminal 3. Alternatively, a system for inputting a heart rate measured by other simplified pulse rate sensor or cardiac sensor in the portable information terminal 3 and processing it by the application programs may be employed.

According to the present invention, a simplified and inexpensive pulse rate sensor or cardiac sensor such as the portable information terminal 3, which is not complicated unlike sensors such as chest band directly fixed on the skin of a person, is defined as “portable heart rate sensor.”

With the system for calculating biological information under exercise load according to the present exemplary embodiment, motion data and heart rate data during exercise of the user 2 by use of a simplified pulse rate sensor or cardiac sensor are collected and analyzed in the server 6 so that the user 2 can know high-accuracy biological information under exercise load such as maximum oxygen uptake (VO₂max) indicating physical strength or the like in the simplified measurement method. Calculation of maximum oxygen uptake (VO₂max) will be described below by way of example, but needless to say, the server 6 can generate and output also biological information other than maximum oxygen uptake based on acceleration data, heart rate data, or the like under exercise load.

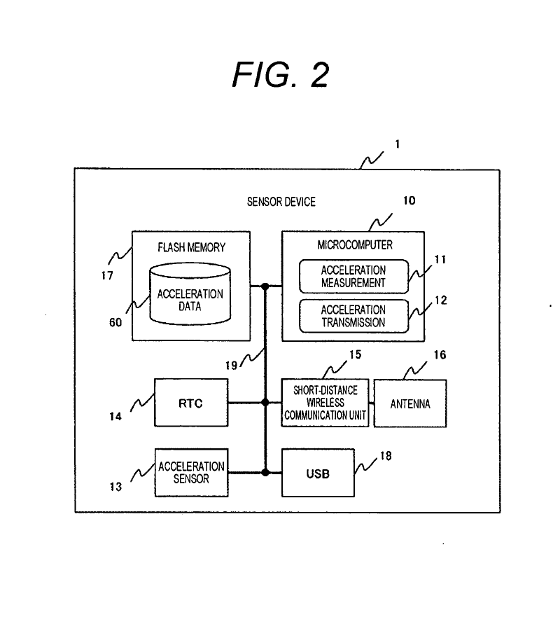

FIG. 2 illustrates main components in the sensor device 1, which is configured of a microcomputer 10, an acceleration sensor 13 capable of measuring a motion or orientation of a person, a RTC (real time clock) 14 for holding current time information or calendar information, a short-distance wireless communication unit 15 conforming to a low-power consumption communication standard such as Bluetooth for communicating with the portable information terminal 3, an antenna 16 for transmitting transmission data from the short-distance wireless communication unit 15 via radio waves, a flash memory as large-capacity nonvolatile storage device, a USB communication unit 18 for communicating with an external device via USB as wired communication means, and a communication line 19 as a device-to-device communication path.

The microcomputer 10 operates according to the programs described in an acceleration measurement program 11 and an acceleration data transmission program 12 which are previously recorded therein. With the acceleration measurement program 11, the microcomputer 10 can acquire the acceleration data 60 as information measured by the acceleration sensor 13 at predetermined intervals, and add time stamps as acceleration measurement time information acquired from the RTC 14 at predetermined intervals, and record it in the flash memory 16. With the acceleration measurement program 11, the microcomputer 10 needs to operate only for measuring an acceleration at predetermined intervals or recording acceleration data into the flash memory 16, thereby restricting consumed power, and for example when measuring at a cycle of more than 20 Hz required for measuring person's daily operations, the microcomputer 10 can operate with a small built-in battery over several days even if it is always operating, and thus maintenance such as battery charging is easy. When the microcomputer 10 measures an acceleration at a similar measurement cycle, the amount of data to be recorded is much smaller than the capacity of a general-purpose flash memory, and the microcomputer 10 easily records over several weeks. With the acceleration data transmission program 12, the microcomputer 10 can automatically determine an arbitrary timing of the user 2 or a timing at which it is communicable with the portable information terminal 3, can control the short-distance wireless communication unit 15 to start communication, and can transmit the acceleration data 60 recorded in the flash memory 16 to the portable information terminal 3. Alternatively, the USB communication unit 19 can detect wired communication with the portable information terminal 3 or the like, and start transmitting the acceleration data 60. Even if transmitted at elapse of a time after measurement, sensor data 17 can be rearranged and recorded in a time sequence in the portable information terminal 3 or the server 6 to receive since it is added with time stamps.

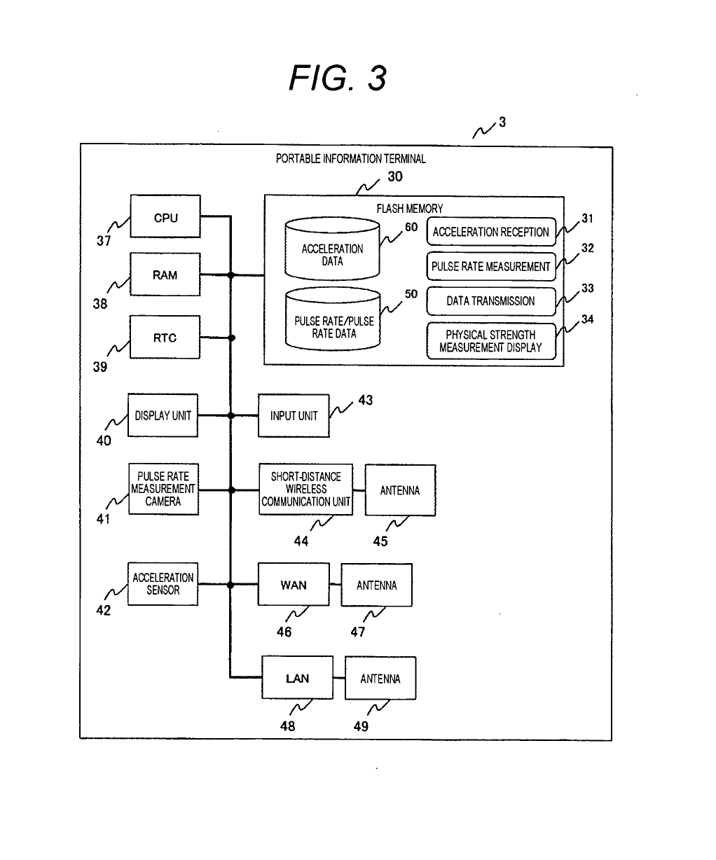

FIG. 3 illustrates main components in the portable information terminal 3, which is configured of a CPU (central processing unit) 37 for executing programs recorded in a flash memory 30 as large-capacity nonvolatile storage device thereby to control other devices, a RTC 39 for holding current time information or calendar information, a display unit 40 capable of displaying arbitrary information designated by a program, an input unit 43 such as keyboard or touch panel operated by a user for operating the programs, a sensor 42 capable of measuring a tilt or motions of the portable information terminal 3, a RAM 38 as random access memory for temporarily recording therein information required for the processings of the CPU 37, a short-distance wireless communication unit 44 conforming to a low-power consumption communication standard such as Bluetooth for communicating with the sensor device 1 or the like, an antenna 45 for transmitting transmission data from the short-distance wireless communication unit 44 via radio waves, a wide-area wireless communication unit 46 connected to the Internet 5 and conforming to a similar communication standard of cell phones for communicating with the server 6 o the like, a wireless LAN communication unit 48 connected to the Internet 5 for communicating with the server 6 or the like via the base station 4 in a short distance such as indoors conforming to the IEEE 802.11 or the like, an antenna 49 for transmitting transmission data from the wireless LAN communication unit 48 via radio waves, a camera 41 configured of a sensor or lens capable of shooting an image, and capable of measuring pulses by continuously shooting a finger or the face, and a communication line 21 as a device-to-device communication path, and is characterized in that acceleration sensor data or the pulse rate data measured or input by the user 2 can be temporarily accumulated in the flash memory 30 and transmitted to the server 6 via the base station 4 connected to the Internet 5 and contents such as display data received from the server 6 can be displayed on the display unit 40. The flash memory 30 stores therein a plurality of programs describing the operations of the portable information terminal 3. With an acceleration data reception program 31, the CPU 37 controls the short-distance wireless communication unit 44 to receive the acceleration data transmitted from the sensor device 1 and to record it as the acceleration data 60 in the flash memory 30. That is, even in an environment in which the CPU 37 cannot always connect to the server 6, the received acceleration data will not be lost. With a pulse rate measurement program 32, the CPU 37 can control the camera 41 to continuously shoot the skin of a finger or the face of the user 2, can detect a periodical change in color or the like of the skin due to blood flow conversion, and can measure and record a pulse rate in the pulse rate data 50 in the flash memory 30. In physical strength measurement according to the present exemplary embodiment, a pulse rate measured via the skin of an acral part of a person is regarded as almost the same as a heart rate which is the number of pulsations of the heart. In pulse rate measurement using an image device such as the camera 41 or a device for detecting a change in intensity of light, a detected noise component irrelevant of pulses due to disturbance light or body motion lowers a measurement accuracy except at rest. Therefore, it is desirable to make measurements not during exercise but after exercise. An input screen is displayed on the display unit 40, and a value input by the user 2 via the input unit 43 can be recorded in the pulse rate data 50 in the flash memory 30. A measurement date/time is acquired from the RTC 39 and added to the pulse rate data 50 for recording, and thus the measurement time can be determined later by the server 6 or the like. When input, the measurement time can be set at an arbitrary time by the user 2. With the data transmission program 32, the CPU 37 controls the wide-area wireless communication unit 46 or the wireless LAN communication unit 48 thereby to transmit the acceleration data 60 and the pulse rate data 50 recorded in the flash memory 30 to the server 6 connected to the Internet 5 via the base station 4. With a physical strength measurement result display program 34, the CPU 37 instructs a procedure of an exercise test such as three-minute walking to the user and can activate other programs by a user operation. Further, the CPU 37 can display a physical strength value calculated based on the acceleration data 60 and the pulse rate data 50 in the server 6, or estimated maximum oxygen uptake (VO₂max) on the display unit 40, and notifies it to the user 2. Maximum oxygen uptake can be estimated by a well-known algorithm such as Astrand nomogram in a relationship between heart rate and exercise amount (or oxygen uptake or consumption energy) in a state in which exercise is being done at a constant pace for at least three minutes or in an aerobic exercise state in which consumed oxygen is balanced with oxygen uptake due to exercise. Further, with the physical strength measurement result display program 34, the CPU 37 displays an interface for inputting body characteristic amount data of the user 2 required to estimate maximum oxygen uptake (VO₂max) on the display unit 40, thereby promoting to input prior to estimating maximum oxygen uptake, and transmitting the input value to the server 6. The body characteristic amount data is height, weight, age, resting heart rate, smoker/non-smoker, and the like, for example. The resting heart rate can be measured similarly with the pulse rate measurement program 32 by the controlled camera 41 to be assumed as an input value. In resting pulse rate measurement, the sensor 42 determines whether the user is at rest for more than a predetermined time or the user has just awakened, and starts the measurement, thereby accurately measuring a resting heart rate. Further, the resting heart rate is likely to have a different value in a different measurement time zone, such as between immediately after wake-up and during daytime, and thus is measured under as identical conditions as possible.

The calculation for estimating maximum oxygen uptake (VO₂max) from the acceleration data and the pulse rate data is desirably made in the high-speed server 6 for reducing a calculation time, but can be made also in the portable information terminal 3 if a calculation time or consumption power of the portable information terminal 3 can be ignored. If consumption power can be similarly ignored, an acceleration measured by the sensor device 1 can be measured by the sensor 42 in the portable information terminal 3.

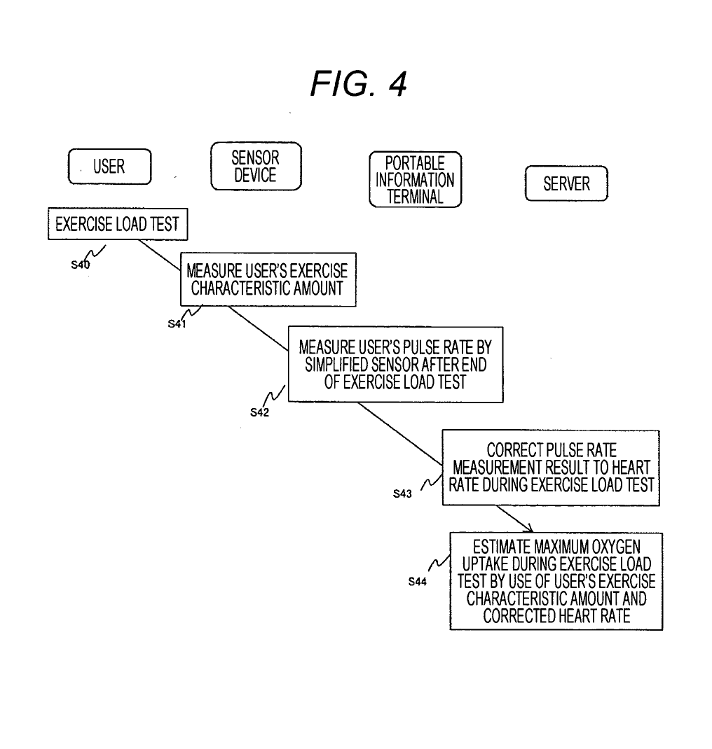

FIG. 4 is a flowchart illustrating an outline of a processing of calculating biological information under exercise load according to the first exemplary embodiment. At first, the user 2 starts a predetermined “exercise load test” (S40). During the exercise load test, the exercise characteristic amount of the user is measured by the sensor device 1 including an acceleration meter (S41). Further, within a predetermined time after the end of the exercise load test, a user's pulse rate is measured by a simplified sensor such as the portable information terminal 3 (S42). The exercise characteristic amount or the pulse rate data is sent to the server 6. The pulse rate measurement result is corrected to a heart rate during the exercise load test by the server 6 (S43). Then, maximum oxygen uptake (VO₂max) of the user during the exercise load test is estimated by use of the exercise characteristic amount of the user and the corrected heart rate (S44).

When a simplified pulse monitor such as the camera 41 in the portable information terminal 3 is used, a pulse rate cannot be accurately measured during user's exercise. Thus, in order to measure a pulse rate during aerobic exercise, the measurement needs to be made at rest when the exercise is stopped (S42). In this case, the pulse rate drops in a temporal difference between the end of the exercise and the measurement of the pulse rate, and is different from the value during the exercise. Thus, with the heart rate correction program 54 in the server 6, the CPU 57 calculates a temporal difference leading to a drop in heart rate based on the walking interval data in the walking data 100 and a heart rate measurement time corresponding to the walking interval recorded in the heart rate data 50, estimates a pulse rate drop based on the temporal difference, and records it in the walking data 100.

The present invention is characterized in that a coefficient for converting a temporal difference into a drop recorded in pulse rate drop data 80 is used for calculating a pulse rate drop. This point will be described below in detail. With the maximum oxygen uptake estimation program 55, the CPU 57 calculates one estimated value of maximum oxygen uptake (VO₂max) per walking interval recorded in the walking data 100, and records it in maximum oxygen uptake data 90. When calculating maximum oxygen uptake (VO₂max), the CPU 57 estimates by regression analysis by use of the exercise characteristic amount and the heart rate (a pulse rate is also recorded as a heart rate) in one walking interval recorded in the walking data 100, and the body characteristic amount 70 of the user 2 to be measured. This is the same principle as the algorithm for estimating maximum oxygen uptake (VO₂max) in a relationship between heart rate and exercise amount, and maximum oxygen uptake can be estimated at a higher accuracy according to the present exemplary embodiment using a plurality of exercise characteristic amounts based on the acceleration data than when the exercise amount is replaced with a walking speed or the like for measurement.

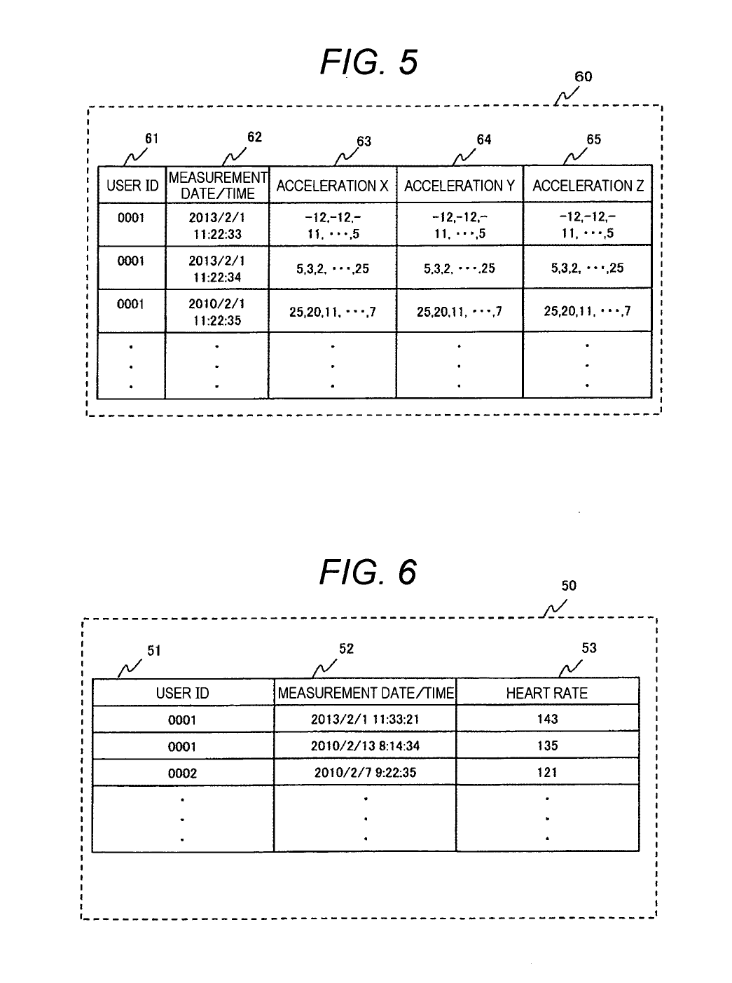

FIG. 5 illustrates a structure of the acceleration data 60. The acceleration data 60 is recorded as time-based data per user in each row. User ID 61 is an identifier indicating each item of data measured for which user. Measurement date/time 62 indicates year/month/day and time when an acceleration is measured. According to the present exemplary embodiment, the measurement date/time 62 is added to the acceleration value per second, and a unit of millisecond or less may be employed. Acceleration X 63, acceleration Y 64, and acceleration Z 65 record the values of three-axis acceleration corresponding to the user ID 61 and the measurement time 62, respectively. The measurement date/time 62 by the second is used to record data at a measurement cycle in millisecond or less, and thus a plurality of measurement values at a corresponding second are recorded in one row in order of measurement. For example, when a value of the acceleration sensor capable of measuring an acceleration of ±4G is read in a resolution of 8 bit, −4G of the acceleration is −128 and 4G thereof is 127.

FIG. 6 illustrates a structure of the heart rate data 50. The heart rate data or the pulse rate data measured or input by the portable information terminal 3 is recorded in each row at each measurement time per user. The definitions of pulse rate and heart rate are generally different, but according to the present exemplary embodiment, pulse rate data is also handled as heart rate data. User ID 51 is an identifier corresponding to a user 2 whose heart rate is measured. Measurement date/time 52 is year/month/day and time when a heart rate is measured. Heart rate 53 is a heart rate or pulse rate measured at measurement time 52 by the user 2 corresponding to the user ID 51. Pulse rate 53 is a value before being corrected by the pulse rate correction program 54.

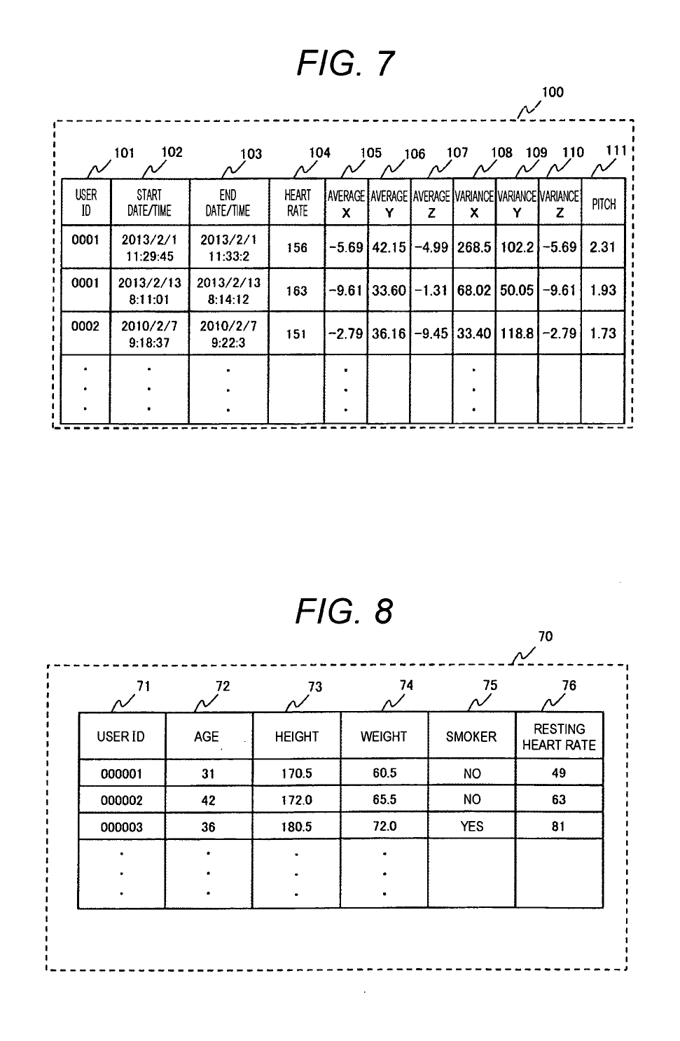

FIG. 7 illustrates a structure of the walking data 100. The walking data 100 records therein data on each interval in which a walking state is detected at a constant pace for more than three minutes per user in each row. User ID 101 indicates an identifier of the user 2 corresponding to a walking interval of each row. Start time 102 indicates year/month/day and time when a detected walking interval starts. End time 103 indicates year/month/day and time when a detected walking interval ends. Heart rate 104 indicate a heart rate corresponding to a walking period, and records therein a result obtained by correcting a resting pulse rate measured after walking by the pulse rate correction program 54 and estimating and correcting a value immediately before the end of the walking. Average X 105, average Y 106, and average Z 107 record therein an average of each axis of three-axis acceleration in a walking interval. Variance X 107, variance Y 108, and variance Z 109 record therein a variance value of each axis of three-axis acceleration in a walking interval. Walking pitch 111 indicates a duration per step detected by a well-known walking detection algorithm from three-axis acceleration of a walking interval.

FIG. 8 illustrates a structure of the body characteristic amount data 70 input by each user, which is recorded in data in each row per user. User ID 71 indicates an identifier of a user corresponding to data of each row. Age 72 indicates the age of a user. Height 73 indicates the height (by the centimeter or by the meter) of a user. Weight 74 indicates the weight (by the kilogram) of a user. Smoker 75 indicates whether a user regularly smokes at present. Resting heart rate 76 indicates a heart rate at complete rest (ideally, while sleeping or on awakening) of a user.

As described above, the present invention is characterized in that a coefficient for converting a temporal difference into a drop recorded in the pulse rate drop data 80 is used for calculating a pulse rate drop. The inventors have found that the property of a pulse rate drop immediately after walking is less different among persons as a result of the investigation.

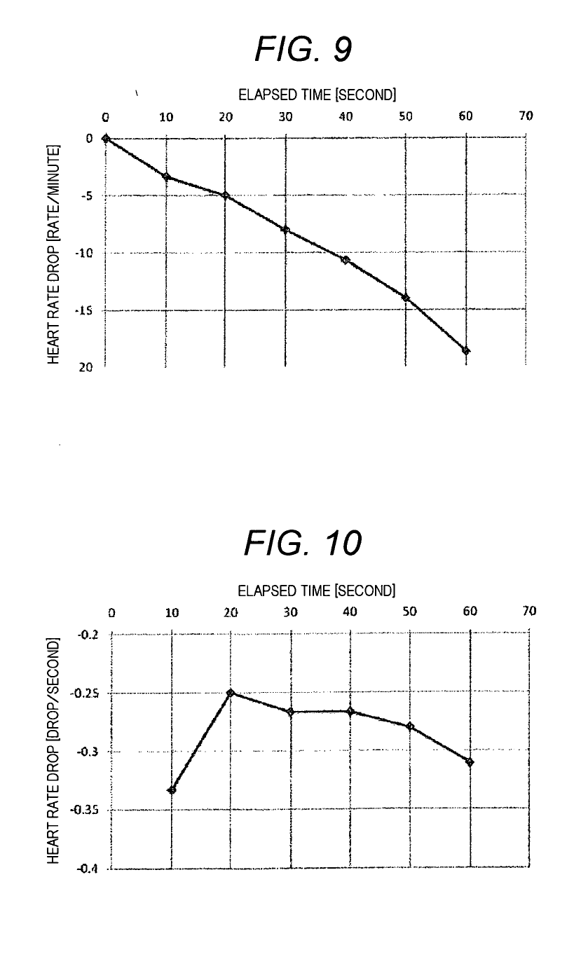

FIG. 9 illustrates a typical change in heart rate after three-minute walking measured for male adults in their 20s to 40s by the inventors. A personal difference in the change within one minute after the stop of walking is less effective on estimation of maximum oxygen uptake (VO₂max).

When a heart rate drop is calculated based on the change, the graph in FIG. 10 is obtained. The heart rate drop data 80 can be created based on the graph of the drop.

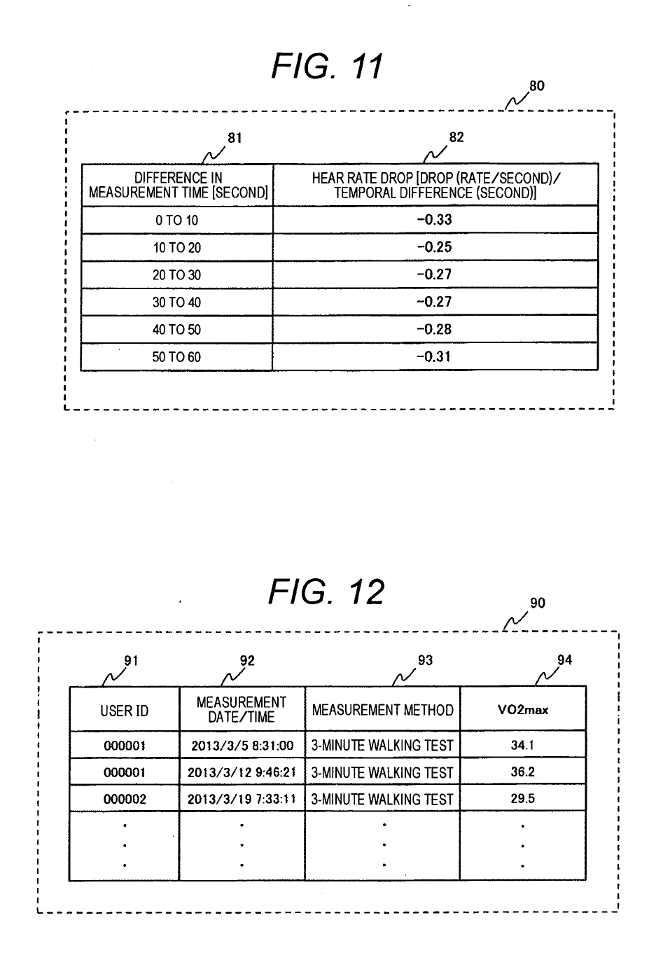

FIG. 11 illustrates a structure of the heart rate drop data 80. The heart rate drop data 80 previously records therein a drop (drop [rate/minute]/temporal difference [second]) indicating a heart rate drop within a time with less personal difference such as within one minute after the stop of most typical exercise and corresponding to a temporal difference between the end of exercise and the measurement of heart rate. The heart rate drop data 80 can be created based on the data on heart rate change after exercise previously collected from a plurality of subjects. The data and table in FIG. 9 to FIG. 11 are made for male adults, and a similar trend can be found for female adults. When the present invention is applied for elderly persons or children, the data of each generation is collected and made in a database, thereby making estimations at a higher accuracy.

The table of the heart rate drop data 80 can be created per user by learning the data measured per user. The coefficients in the table are multiplied with a time between the stop of exercise and the measurement of heart rate, thereby estimating a heart rate drop per user at a higher accuracy.

FIG. 12 illustrates a structure of the maximum oxygen uptake data 90, in which a measurement value per user is recorded in each row. When a heart rate is measured and recorded after walking in a walking interval in which a constant pace is detected for more than three minutes, a value of the maximum oxygen uptake (VO₂max) is estimated and recorded. User ID 91 indicates an identifier of a user corresponding to maximum oxygen uptake in each row. Measurement date/time 92 records therein date/time when maximum oxygen uptake (VO₂max) is measured, or the end date/time of a walking interval when estimated from the walking data 100. When measured in another method, the measurement date/time is recorded. Measurement method 93 indicates a maximum oxygen uptake measurement method in each row. That is, data measured in other method can be recorded, not limited to the estimation using the walking data 100. Maximum oxygen uptake 94 records therein data on maximum oxygen uptake (VO₂max: in a unit of milliliter /kilogram/minute) estimated based on the walking data 100 or measured in another means.

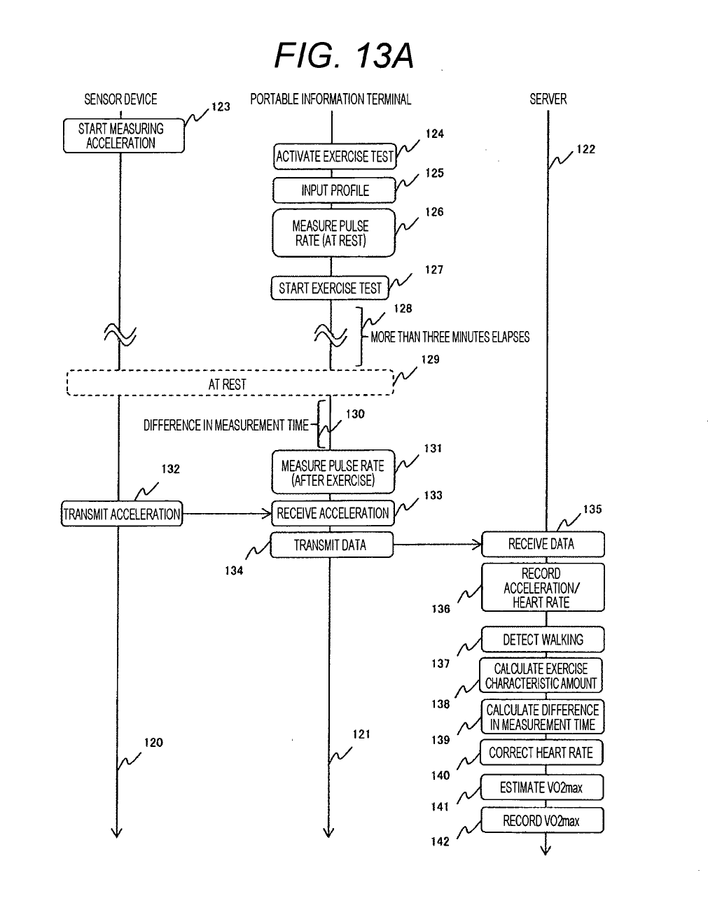

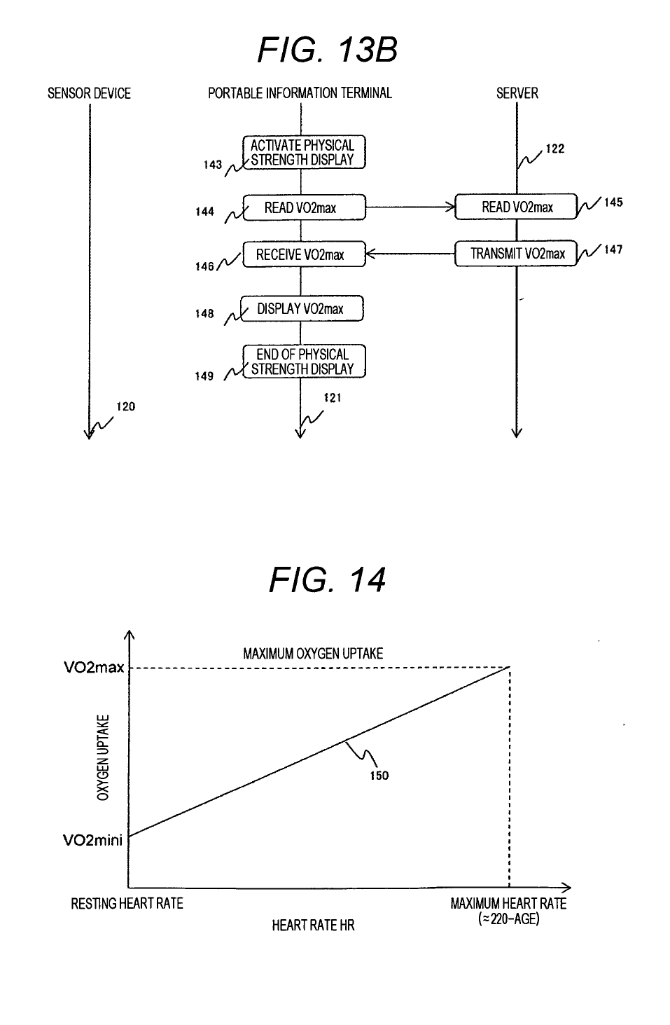

FIG. 13A and FIG. 13B illustrate a procedure of measuring maximum oxygen uptake (VO₂max) according to the present exemplary embodiment. A processing procedure 120, a processing procedure 121, and a processing procedure 122 indicate the processing procedures of the sensor device 1, the portable information terminal 3, and the server 6, respectively. In a processing 123, the sensor device 1 starts measuring an acceleration. The sensor device 1 can always measure an acceleration irrespective of measurement of maximum oxygen uptake, and thus starts measuring prior to measuring maximum oxygen uptake. In a processing procedure 124, the physical strength measurement display program 34 in the portable information terminal 3 is activated to display a procedure or menu required for measuring maximum oxygen uptake. In a processing 125, the user inputs the body characteristic amount only when not having input it or requiring correction. In a processing 126, a resting heart rate capable of being input also in the processing 125 is measured by the pulse rate measurement program 54 in the portable information terminal 3. When measuring a resting heart rate, a rest state of more than a predetermined time is detected by the sensor 42 in the portable information terminal 3, and then a pulse rate is measured.

In a processing 127, the physical strength measurement display program 34 in the portable information terminal 3 is operated to start an exercise test of three-minute walking, and a lapse of three minutes can be measured by a timer and notified. On the other hand, without such a navigation to the user, maximum oxygen uptake (VO₂max) can be estimated by walking data at a constant pace for more than three minutes and heart rate data after walking. A walking interval 128 needs more than three minutes for acquiring an aerobic exercise state in which consumed oxygen by exercise is balanced with oxygen uptake.

When the timer counts more than three minutes after the start of the exercise test, the user stops the exercise test, enters the rest state (rest interval 129), and starts measuring a pulse rate. A measurement temporal difference 130 is a time after the user 2 actually stops the exercise and transits to the rest state and before the user starts measuring a pulse rate. In a processing 131, the user operates the physical strength measurement display program 34 to measure a pulse rate. The user uses the camera 41 provided in the portable information terminal to measure a pulse rate after exercise based on an instruction of the display unit in the portable information terminal 3. Alternatively, a value measured by another simplified heart rate sensor or pulse rate sensor may be input without the use of the camera 41 or the like.

In a processing 132, the acceleration data including the walking interval 128, which is measured and recorded by the sensor device 1 in asynchronism with the exercise test of three-minute walking in a state in which the sensor device 1 is communicable with the portable information terminal 3, is transmitted to the portable information terminal 3. In a processing 133, the portable information terminal 3 receives the data transmitted from the sensor device 1 and records it in the flash memory 30. In a processing 134, the portable information terminal 3 transmits the acceleration data 60 and the pulse rate data 50 recorded in the flash memory 30 to the server 6 in asynchronism with the processing 133 in the communicable state with the server 6.

The server 6 receives the data transmitted from the portable information terminal 3 in a processing 135, and records it in the storage 36 in a processing 136. In a processing 137, with the walking detection program 52 in the server 6, the walking interval 128 of more than three minutes is detected from the received acceleration data 60 and is recorded in the walking data 100. In a processing 138, with the exercise characteristic amount calculation program 53, the exercise characteristic amount corresponding to the walking interval 128 recorded in the walking data 100 is calculated and recorded in the walking data 100. In a processing 139, with the heart rate correction program 54, a heart rate measured within one minute after the end of the walking interval 128 recorded in the walking data 100 is searched from the heart rate data 50, and a difference between the end date/time of the walking interval 128 and the measurement date/time of the heart rate is calculated as the measurement temporal difference 130. In a processing 140, with the heart rate correction program 54, a heart rate drop is estimated by use of the heart rate drop data 80 based on the measurement temporal difference 130 calculated in the processing 139, and a value of the heart rate data 50 added with the value of the drop is recorded in the heart rate 104 in the walking data 100. In a processing 141, with the maximum oxygen uptake estimation program 55, maximum oxygen uptake (VO₂max) is estimated by regression analysis by use of the walking data 100 corresponding to the walking interval 128 of the exercise test of three-minute walking and the body characteristic amount data 70 of the user 2 under measurement. In a processing 142, the result estimated in the processing 141 is recorded in the maximum oxygen uptake (VO₂max) data 90.

There will be described below the processings when the user wants to know maximum oxygen uptake acquired by his/her exercise test. In a processing 143, the user activates the physical strength display program on the display unit in the portable information terminal 3. Accordingly, in processings 144 to 147, the portable information terminal 3 acquires the maximum oxygen uptake data recorded in the server 6, and in processings 148 to 149, the information is displayed on the display unit in the portable information terminal 3 and the user confirms it and terminates.

FIG. 14 is a diagram illustrating a relationship between oxygen uptake VO₂ and heart rate HR, which is a graph illustrating a typical principle of measuring maximum oxygen uptake (VO₂max). In order to actually measure maximum oxygen uptake, oxygen uptake at a maximum heart rate of a subject needs to be measured by an expired gas analyzer. A regression line 150 using a correlation relationship between oxygen uptake and heart rate is used for simplified measurement. It is used to measure oxygen uptake at a maximum heart rate or less and to estimate a value at the maximum heart rate according to the regression line 150. It is said that the maximum heart rate can be estimated in 220−age. VO₂mini is oxygen uptake not at rest. When an expired gas analyzer is not used, oxygen uptake is estimated based on the amount of exercise during the exercise. The amount of exercise can be estimated by a well-known algorithm based on a walking speed while walking, for example. With the maximum oxygen uptake estimation program 55 according to the present exemplary embodiment, a higher-accuracy estimation can be made by multi-regression analysis using a plurality of exercise characteristic amounts capable of being calculated based on acceleration data different from a walking speed.

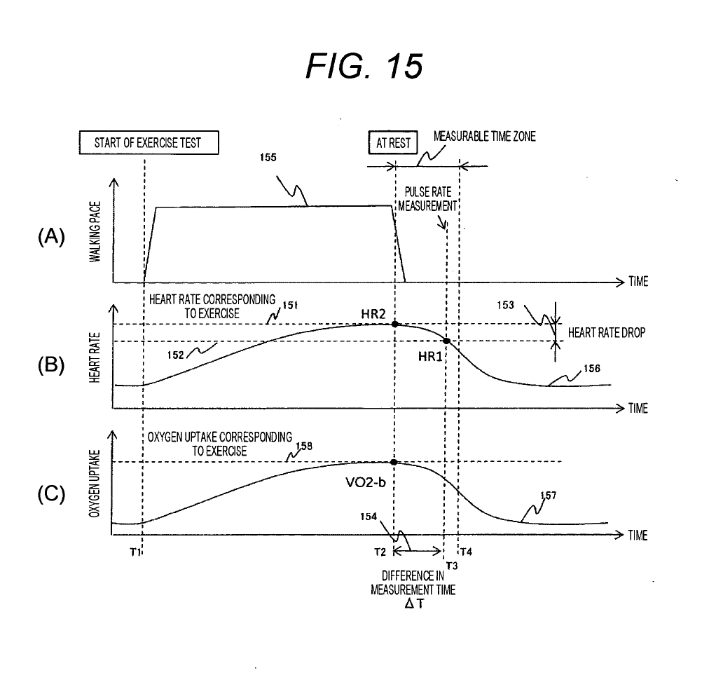

FIG. 15 is a graph illustrating a principle of correcting a heart rate according to the present exemplary embodiment. FIG. 15 illustrates, as an example of the amount of exercise, an example in which the user walks at a constant pace only for a predetermined time (the walking interval 128 in FIG. 13A) and a pulse rate is measured immediately thereafter (after the rest interval 129 in FIG. 13A). The start time of an exercise load test, the end (transition to rest state) time, and a pulse rate measurable time zone, and an actual measurement time are assumed as T1, T2, T2−T4, and T3, respectively. When a walking pace 155 is constantly continued for the exercise load test as illustrated in FIG. 15(A), a heart rate 156 of the user increases and is generally saturated at a heart rate 151 corresponding to the exercise of more than three minutes as illustrated in FIG. 15(B). As illustrated in FIG. 15(B), oxygen uptake 157 is also saturated at oxygen uptake 158 corresponding to the exercise. Maximum oxygen uptake (VO₂max) can be estimated by the principle of the regression line 150 by use of a value of the heart rate 151 corresponding to the exercise or a value HR2 of the heart rate immediately before the stop of the exercise load test (time T2) and a value of the oxygen uptake 158 corresponding to the exercise, or a value VO₂b of oxygen uptake immediately before the stop of the exercise load test (time T2). The value VO₂b of the oxygen uptake 157 can be measured by an always-measurable device such as acceleration sensor, but the value HR2 of the heart rate 156 cannot be accurately measured during exercise by an inexpensive and simplified pulse rate sensor or the like. Therefore, the measurement is made after exercise. In this case, a measurement temporal difference 154 is caused between T2 immediately before the stop of the exercise load test and the actual measurement time T3, and thus the heart rate for the heart rate drop 153 lowers than the value HR2 of the heart rate 151 corresponding to the exercise to be the value HR1 of the heart rate 152 on measurement, and maximum oxygen uptake cannot be accurately estimated.

According to the present exemplary embodiment, the user measures the value HR1 of the heart rate 152 at time T3 after exercise by use of the camera 41 in the portable information terminal 3. Then, with the heart rate correction program 54 in the server 6, the measured value HR1 of the heart rate 152 is corrected to an estimated value HR2 of the heart rate 151 corresponding to the exercise based on the measurement temporal difference 154, which enables maximum oxygen uptake (VO₂max) to be accurately estimated. As described above, if the measurement temporal difference 154 is within about one minute, the heart rate drop 153 is less different among persons, and an accurate correction based on previously-acquired data is enabled. Thus, the measurable time zone T2−T4 is set within one minute.

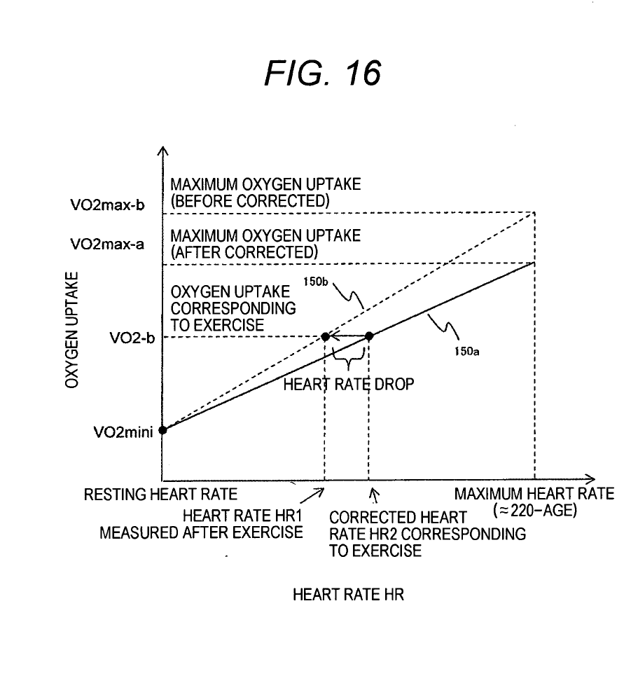

FIG. 16 is a graph illustrating a principle of estimating maximum oxygen uptake (VO₂max) according to the first exemplary embodiment. A regression line 150b is acquired based on the value HR1 of the heart rate 152 after exercise, the value VO₂b of the oxygen uptake 157 immediately before the stop of exercise, and the minimum oxygen uptake at rest, which are measured by the user. According to the regression line 150b, the maximum oxygen uptake of the user is VO₂max−b. However, if the estimated value HR2 of the heart rate 151 during exercise, which is corrected by the heart rate correction program 54, is employed, a regression line 150a is acquired based on the estimated value, the value VO₂b of the oxygen uptake 157 during exercise, and the minimum oxygen uptake at rest. According to the corrected regression line 150a, the maximum oxygen uptake of the user is VO₂max−a. The value VO₂max−a is an estimated value of the maximum oxygen uptake of the user.

In FIG. 14 and FIG. 16, one regression line is basically employed, but a plurality of regression lines or regression curves corresponding to user's habitual smoking, age group, and the like may be employed as a finer estimation system based on the user profile.

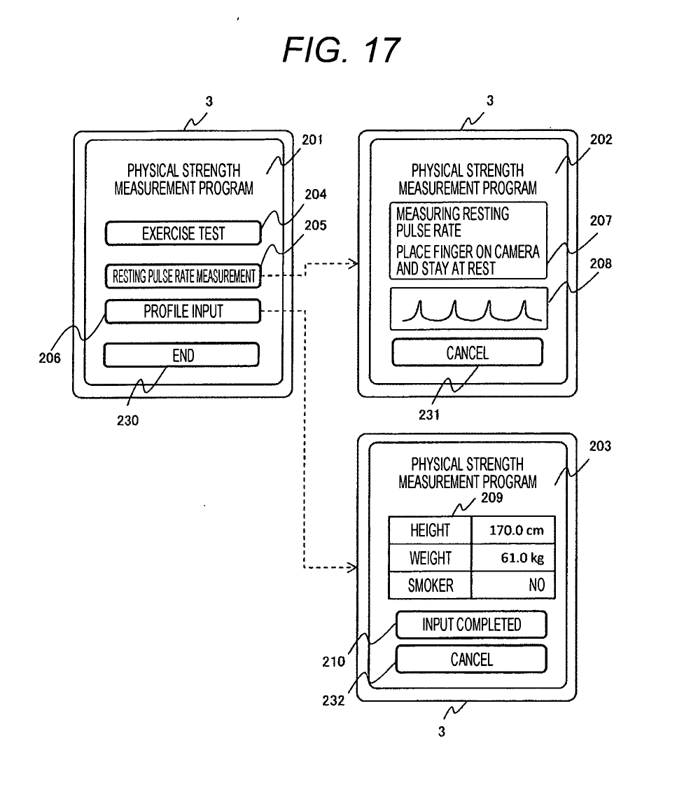

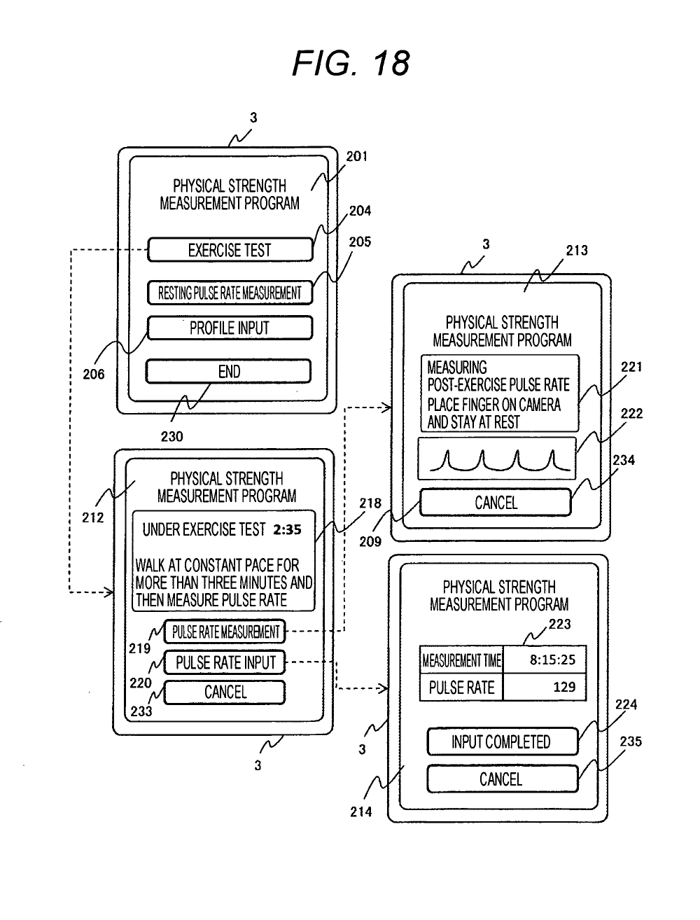

FIG. 17 and FIG. 18 illustrate exemplary screen display contents on the display unit 40 in the portable information terminal 3 during physical strength measurement. At first, in FIG. 17, when the user activates the physical strength measurement display program 34, an initial screen 201 is displayed. The user selects an exercise test start button 204, starts the timer of three minutes thereby to start an exercise test, and transits to exercise test screen display 212. The user selects a resting pulse rate measurement button 205 thereby to transit to a resting pulse rate measurement screen 202. The user selects a profile input button 206 thereby to transit to a profile input screen 203. The user selects an end button 230 thereby to terminate the physical strength measurement display program 34. The resting pulse rate measurement screen 202 displays therein measurement procedure display 207 and pulse wave display 208, a rest state of more than a predetermined time is detected, and a pulse rate is measured. When a pulse rate is completely measured, the measured pulse rate is transmitted as a resting heart rate to the server 6, and the resting pulse rate measurement screen 202 returns to the initial screen 201. Alternatively, the user selects a cancel button 231 thereby to return to the initial screen 201. The user 2 can input height, weight, and smoker/non-smoker in an input form 209 on the profile input screen 203, selects an input complete button 210 after input thereby to transmit the input contents as the body characteristic amount of the user 2 to the server 6, and returns to the initial screen 201. Alternatively, the user selects a cancel button 232 thereby to return to the initial screen 201.

Then, in FIG. 18, when the user selects the exercise test start button 204 on the portable information terminal 3 to start an exercise test, the exercise test screen display 212 displays thereon the procedure of the exercise test or an elapsed time after the start in exercise test procedure display 218. When the user selects a pulse rate measurement button 219 after walking of more than three minutes thereby to transit to a post-exercise pulse rate measurement screen 213. Since the post-exercise pulse rate measurement needs to be performed in a limited time zone such as within one minute after the stop of the exercise, it is desirable to additionally display a pulse rate measurable time zone (time T2−end time T4) and a current time in the time zone to be notified to the user on the exercise test screen display 212 or the post-exercise pulse rate measurement screen 213.

The user selects a pulse rate input button 220 to transit to a post-exercise pulse rate input screen 214 including an input form 223. Alternatively, the user selects a cancel button 233 to return to the initial screen 201. The post-exercise pulse rate measurement screen 213 displays therein post-exercise pulse rate measurement procedure display 221 and pulse wave display 222 to measure a pulse rate of the user, and when completing the measurement of a pulse rate, records the pulse rate in the flash memory and transmits it to the server 6, and returns to the initial screen 201. Alternatively, the user selects the cancel button thereby to return to the initial screen 201. The user can input a pulse rate measured by another instrument and a pulse rate measurement time in the input form 223 in the post-exercise pulse rate input screen 214. The user selects an input complete screen 224 after input thereby to transmit the input pulse rate and measurement time to the server 6 as measured in the post-exercise pulse rate measurement screen 213, and returns to the initial screen 201. Alternatively, the user selects a cancel button 235 thereby to return to the initial screen 201.

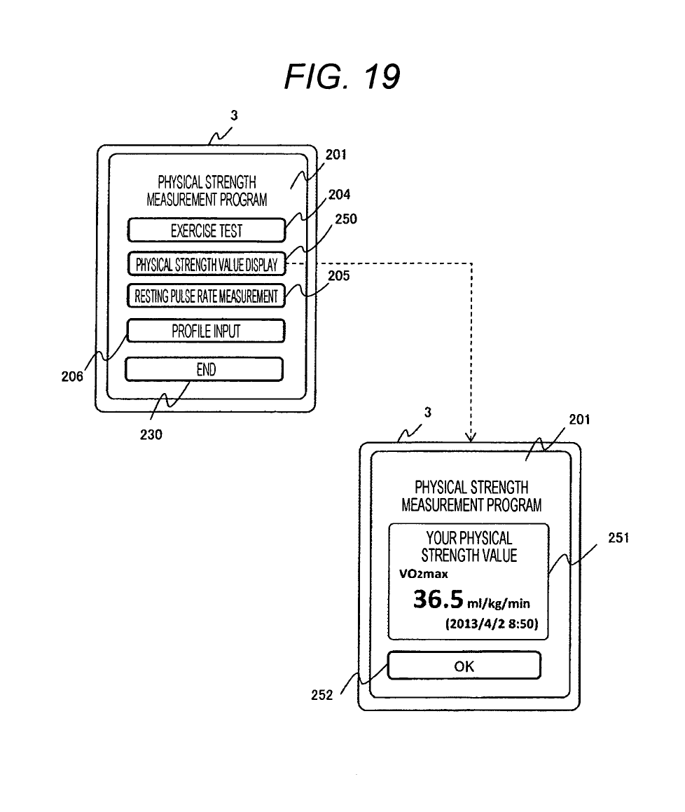

FIG. 19 illustrates an example in which the physical strength measurement display program 34 includes a physical strength value display function. A physical strength value display button 250 present on the initial screen 201 in the portable information terminal 3 is selected thereby to transit to a physical strength value display screen. The physical strength value display screen displays thereon an estimated value (VO₂max−a) of maximum oxygen uptake of the user as VO₂max 251 based on the corrected regression line 150a. An OK button 252 is selected thereby to return to the initial screen 201. In this way, the user can easily acquire a heart rate during exercise, and consequently accurate information on biological information under exercise load by use of an inexpensive and simplified pulse rate sensor or cardiac sensor such as a portable information terminal.

There has been described in the first exemplary embodiment the example in which maximum oxygen uptake (VO₂max) is estimated as biological information under exercise load, but the present invention is not limited thereto and other biological information of the user during exercise can be acquired. For example, calorie consumption can be also estimated based on the value VO₂b of the oxygen uptake 157 during user exercise and the weight of the user.

### REFERENCE SIGNS LIST

1 sensor device
2 user
3 portable information terminal
4base station
5 Internet
6 server
7 program memory
8 storage
11 acceleration measurement program
12 acceleration data transmission program
14 read time clock (RTC)
18 universal serial bus (USB) communication unit
20 program memory
31 acceleration data reception program
32 pulse rate measurement program
33 data transmission program
34 physical strength measurement display program
36 storage
37 central processing unit (CPU)
38 random access memory (RAM)
39 read time clock (RTC)
46 wide-area wireless communication unit
48 wireless local area network (LAN) communication unit
50 pulse rate data
51 data reception program
52 walking detection program
53 exercise characteristic amount calculation program
54 heart rate correction program
55 maximum oxygen uptake estimation program
57 central processing unit (CPU)
58 random access memory (RAM)
59 local area network (LAN) communication unit
60 acceleration data
120 processing procedure for sensor device
121 processing procedure for portable information terminal
122 processing procedure for server
123 to 127 processing
128 exercise test time
128 rest operation
129 to 142 processing
150 regression line
152 post-exercise heart rate
155 walking pace
156 heart rate
157 oxygen uptake
201 initial screen
202 resting pulse rate measurement screen
203 profile input screen
204 to 206 button display
207 instruction display
208 pulse waveform display
209 input form display
210 button display
212 exercise test screen
213 post-exercise pulse rate measurement screen
214 post-exercise pulse rate input screen
219 button display
220 button display
221 instruction display
222 pulse waveform display
223 input form
224 to 235 button display
250 physical strength value display button

1. A system for calculating biological information under exercise load, comprising:
a sensor device for measuring a motion of a user;
a portable heart rate sensor for measuring a heart rate of a user; and
a server comprising a biological information calculation function,
wherein the system:
measures a motion of a user under exercise load on the user;
measures the heart rate of the user after the stop of the exercise load;
measures a temporal difference between the stop of the exercise load and measurement of the heart rate;
estimates a drop in the heart rate based on the temporal difference and finds an estimated heart rate immediately before the stop of exercise of the user; and
calculates biological information of the user under the exercise load based on the motion of the user under the exercise load and the estimated heart rate.

2. The system for calculating biological information under exercise load according to claim 1,
wherein the server comprises heart rate drop data corresponding to a temporal difference between the stop of the exercise load and measurement of the heart rate in order to estimate a drop in the heart rate based on the temporal difference.

3. The system for calculating biological information under exercise load according to claim 2,
wherein a time zone in which the heart rate is measurable is within one minute after the stop of the exercise load.

4. The system for calculating biological information under exercise load according to claim 2,
wherein the heart rate drop data is created based on post-exercise heart rate change data previously collected from a plurality of subjects.

5. The system for calculating biological information under exercise load according to claim 2,
wherein the heart rate drop data is created per user by learning data previously measured per user.

6. The system for calculating biological information under exercise load according to claim 2,
wherein the portable heart rate sensor is a portable terminal comprising a built-in camera, and
continuously shoots the skin of a finger or the face of the user by the camera, and detects a periodical change in color of the skin due to blood flow conversion thereby to measure the heart rate.

7. The system for calculating biological information under exercise load according to claim 2,
wherein the sensor device comprises an acceleration sensor capable of measuring a motion or an orientation of a person, and an RTC for holding current time information or calendar information.

8. The system for calculating biological information under exercise load according to claim 7,
wherein the server comprises:
a heart rate correction means for correcting a measurement result of the heart rate after the stop of the exercise load into a heart rate under the exercise load based on the heart rate drop data and the temporal difference;
an exercise characteristic amount calculation means for calculating the exercise characteristic amount of the user under the exercise load based on a motion of the user measured by the sensor device; and
a biological information estimation means for estimating biological information of the user under an exercise load test by regression analysis by use of the exercise characteristic amount of the user and the corrected heart rate.

9. The system for calculating biological information under exercise load according to claim 8,
wherein the biological information estimation means is a maximum oxygen uptake estimation means, and
the portable heart rate sensor is a portable terminal, and has a function of displaying the maximum oxygen uptake on a screen in response to a request of the user.

10. The system for calculating biological information under exercise load according to claim 6,
wherein the portable terminal has a function of detecting a rest state by a sensor, and instructing to measure a resting heart rate on a screen.

11. A method for calculating biological information under exercise load by a system for calculating biological information,
wherein the system for calculating biological information comprises:
a sensor device for measuring a motion of a user;
a portable heart rate sensor for measuring a heart rate of a user; and
a server comprising a biological information calculation function,
the method comprising the steps of:
measuring a motion of a user under exercise load on the user;
measuring the heart rate after the stop of the exercise load of the user;
measuring a temporal difference between the stop of the exercise load and measurement of the heart rate;
estimating a drop in the heart rate based on the temporal difference and finding an estimated heart rate immediately before the stop of exercise of the user; and
calculating biological information of the user under the exercise load based on the motion of the user under the exercise load and the estimated heart rate.

12. The method for calculating biological information under exercise load according to claim 11, comprising the step of:
estimating a drop in the heart rate from the temporal difference based on heart rate drop data corresponding to a temporal difference between the stop of the exercise load and measurement of the heart rate previously collected from a plurality of subjects.

13. The method for calculating biological information under exercise load according to claim 11,
wherein a time zone in which the heart rate is measurable is within one minute after the stop of the exercise load.

14. A portable terminal connectable to a server via a network, the portable terminal comprising:
a function of communicating with a sensor device for measuring a motion of a user;
a physical strength measurement display function;
a function of measuring a heart rate of a user;
a built-in camera;
a memory; and
a display unit,
wherein a procedure of an exercise load on a user is displayed on the display unit by the physical strength measurement display function,
the camera is controlled to measure heart rates of the user at rest and after the stop of the exercise load and to record the results in the memory by the heart rate measurement function,
a motion of the user under the exercise load measured by the sensor device is accumulated in the memory;
data on a heart rate of the user and a motion of the user is transmitted to the server; and
biological information of the user under the exercise load calculated by the server is acquired and displayed on the display unit.

15. The portable terminal according to claim 14,
wherein a time zone in which the heart rate is measurable is within one minute after the stop of the exercise load, and
a relationship between the time zone in which the heart rate is measurable and current time in the time zone is displayed on the display unit.
